(19) United States
(12) Patent Application Publication
(10) Pub. No.: US 2016/0206333 A1
(43) Pub. Date: Jul. 21, 2016

(54) Allergy Skin Test Devices with Surrounding Pain-Reduction Structures

(71) Applicants: Ziv Harish, Tenafly, NJ (US); Russ Weinzimmer, Milford, NH (US)

(72) Inventors: Ziv Harish, Tenafly, NJ (US); Russ Weinzimmer, Milford, NH (US)

(21) Appl. No.: 14/599,799

(22) Filed: Jan. 19, 2015

(51) Int. Cl. A61B 17/20; A61B 5/00

(52) U.S. Cl. CPC A61B 17/205; A61B 5/411

(57) ABSTRACT

An allergy skin testing device is disclosed that includes at least one central post having at least one sharp probe extending longitudinally from an end of the at least one central post, the at least one sharp probe being for administering an allergen to skin. For each central post, at least three dull probes substantially surround the at least one sharp probe, creating a “pain gate fence”, the at least three dull probes being for activating a pain gate effect affecting nerves of the skin while the at leat one sharp probe administers the allergen to the skin. The at least three dull probes extend further along the central post than the at least one sharp probe, so when all the probes are pressed against the skin using a gripping portion, the plurality of dull probes press into the skin before the at least one sharp probe penetrates the skin.

### FIELD OF THE INVENTION

This invention relates generally to allergy skin test devices, and particularly to allergy skin test devices with structures to reduce pain.

### BACKGROUND OF THE INVENTION

Traditional allergy skin testing devices inflict pain while introducing allergens to the skin using sharp probes. According to pain-gate theory, applying dull pressure shortly before the sharp probes penetrate the sink to introduce the allergens reduces perception of the pain caused by the sharp probes. Reduced-pain allergy skin testing devices employing pain-gate theory to allergy skin testing were first taught by Harish et al. For example, Harish et al first taught using a single dull probe for each plurality of sharp probes. Heine et al then taught a version of this approach using a single dull probe for each plurality of surrounding sharp probes. However, each single dull probe of Heine et al does not provide an acceptable level of pain reduction for the corresponding plurality of surrounding sharp probes.

### SUMMARY OF THE INVENTION

According to the invention, at least one sharp probe is substantially surrounded by at least three dull probes supported by a surrounding collar, or a surrounding collar having a flat end or a bumpy end. The at least three dull probes, or the flat end, or the bumpy end, provides a surround-effect that creates a “pain-gate fence” substantially surrounding an area of sharp probe penetration that would be painful if not blocked by the surrounding pain-gate fence. The pain-gate fence is also referred to as PAIN FENCE™ technology. A pain-gate fence is any arrangement of pain-gates that substantially block or inhibit pain signals that tend to not be blocked by a single dull probe. The surrounding collar and/or the dull probes also tend to hide the sharp probes surrounded thereby, as like being hidden behind a fence, which makes the device of the invention less intimidating and less upsetting to children. By avoiding emotionally upsetting a child to be tested by the device, pain perception is further reduced beyond the physiological “pain gate” effect caused by the surrounding dull probes. Further the physician or nurse administering the test can tell the patient to expect “dull pressure”, such as by suggesting “you will feel dull pressure”. Since the patient only sees dull probes, and has been told to expect “dull pressure”, he/she fully expects to feel dull pressure, and so is even less likely to perceive any sharp penetration of one or more sharp probes, and thus less likely to experience any discomfort.

One general aspect of the invention is an allergy skin testing device that includes a central post having at least one sharp probe extending longitudinally from an end of the central post, the at least one sharp probe being for administering an allergen to skin. At least three dull probes substantially surround the at least one sharp probe, the dull probes being for activating a pain gate effect in nerves of the skin while the at leat one sharp probe administer the allergen to the skin. The dull probes extend further along the central post than the at least one sharp probe extend beyond the central post. A gripping portion of the central post is for securely grasping the central post so as to facilitate pressing the plurality of dull probes into the skin before pressing the at least one sharp probe into the skin using one continuous movement of the central post towards the skin.

In some embodiments, the dull probes are supported by a support structure that is supported by a longitudinal portion of the central post.

In some embodiments, the plurality of sharp probes is arranged in a regularly spaced array.

In some embodiments, the allergy skin testing device further includes a collar in coaxial relationship with the central post, the dull probes being supported by the collar, the collar being resiliently supported by the central post. In further embodiments, the collar is supported by a plurality of resilient members that extend from the central post to the collar. In other further embodiments, there is at least one discontinuity in the collar.

In some embodiments, the allergy skin testing device further includes a collar, wherein the dull probes are formed as bumps on a forward edge of the collar. In further embodiments, the collar is resiliently supported by the central post. In other further embodiments, the collar is rigidly supported by the central post.

In some embodiments, the dull probes are supported by a flange of the central post, the flange having a surface that is substantially co-planar with the end of the central post. In further embodiments, the dull probes are evenly spaced around the periphery of the flange.

Another general aspect of the invention is an allergy skin testing device that includes a central post having a plurality of sharp probes extending longitudinally from an end of the central post, the sharp probes being for administering an allergen to skin. A collar substantially surrounding the plurality of sharp probes has a plurality of dull probes being for activating a pain gate effect in nerves of the skin while the sharp probes administer the allergen to the skin, the dull probes extending further along the central post than the sharp probes extend beyond the central post. A gripping portion of the central post is for securely grasping the central post so as to facilitate pressing the plurality of dull probes of the collar into the skin before pressing the plurality of sharp probes into the skin using one continuous movement of the central post towards the skin.

In some embodiments, the collar is rigidly supported by the central post.

In some embodiments, the collar is resiliently supported by the central post.

In some embodiments, the collar is supported by a flange of the central post, the flange having a surface that is substantially co-planar with the end of the central post.

In some embodiments, the plurality of sharp probes is arranged in a regularly spaced array.

Another general aspect of the invention is an allergy skin testing device that includes a central post having a plurality of sharp probes extending longitudinally from an end of the central post, the sharp probes being for administering an allergen to skin, and having a flange at the end of the central post, the flange having a plurality of dull probes substantially surrounding the plurality of sharp probes, the dull probes being for activating a pain gate effect in nerves of the skin while the sharp probes administer the allergen to the skin. The dull probes extend further along the central post than the sharp probes extend beyond the central post. A gripping portion of the central post is for securely grasping the central post so as to facilitate pressing the plurality of dull probes of the flange into the skin before pressing the plurality of sharp probes into the skin using one continuous movement of the central post towards the skin.

In some embodiments, the plurality of sharp probes is arranged in a regularly spaced array.

In some embodiments, the device further includes a collar supported by the flange, the collar having the plurality of dull probes evenly spaced around the circumference of the collar. In further embodiments, the dull probes are regular undulations running evenly around the circumference of the collar.

In another general embodiment of the invention, a multiple allergy skin testing device includes a plurality of central posts, each post having a plurality of sharp probes extending longitudinally from an end of each central post, the sharp probes being for administering an allergen to skin. A collar substantially surrounds each plurality of sharp probes, each collar having a plurality of dull probes being for activating a pain gate effect in nerves of the skin while the sharp probes administer the allergen to the skin. The dull probes extend further along each central post than the sharp probes extend beyond the central post. A gripping element connected to each central post, the gripping element being for securely controlling the plurality of central post so as to facilitate pressing each respective plurality of dull probes into the skin before pressing each respective plurality of sharp probes into the skin using one continuous movement of the gripping portion towards the skin.

In some embodiments, each collar is rigidly supported by a respective central post.

In some embodiments, each collar is resiliently supported by a respective central post.

In some embodiments, each collar is supported by a respective flange of a respective central post, the flange having a surface that is substantially co-planar with the end of the respective central post.

In some embodiments, each plurality of sharp probes is arranged in a regularly spaced array.

In another general aspect of the invention, an allergy skin testing device includes a central post having a plurality of sharp probes extending longitudinally from an end of the central post, the sharp probes being for administering an allergen to skin. A flat-ended collar having a continuous flat dull edge substantially surrounds the plurality of sharp probes, the flat-ended collar being for activating a pain gate effect in nerves of the skin while the sharp probes administer the allergen to the skin. The flat-ended collar extends further along the central post than the sharp probes extend beyond the central post. A gripping portion of the central post is for securely grasping the central post so as to facilitate pressing the flat-ended collar into the skin before pressing the plurality of sharp probes into the skin using one continuous movement of the central post towards the skin.

In some embodiments, the flat-ended collar is supported by a support structure that is supported by a longitudinal portion of the central post.

In some embodiments, the plurality of sharp probes is arranged in a regularly spaced array.

In some embodiments, the flat-ended collar is resiliently supported by the central post.

In some embodiments, the flat flat-ended collar is rigidly supported by the central post.

In some embodiments, the flat-ended collar is supported by a plurality of members that extend from the central post to the flat-ended collar.

In some embodiments, there is at least one discontinuity in the flat-ended collar.

In some embodiments, the flat-ended collar is coaxially supported by the central post.

In some embodiments, the flat-ended collar is supported by a flange of the central post.

Another general aspect of the invention is a multiple allergy skin testing device including a plurality of central posts, each central post having a plurality of sharp probes extending longitudinally from an end of each central post, the sharp probes being for administering an allergen to skin. A flat-ended collar having a continuous flat dull edge substantially surrounds each plurality of sharp probes, each flat-ended collar being for activating a pain gate effect in nerves of the skin while the sharp probes administer the allergen to the skin. The flat-ended collar extends further along each central post than the sharp probes extend beyond the central post. A gripping element connected to each central post is for securely controlling the plurality of central posts so as to facilitate pressing each respective flat-ended collar into the skin before pressing each respective plurality of sharp probes into the skin using one continuous movement of the gripping portion towards the skin.

In some embodiments, each flat-ended collar is rigidly supported by a respective central post.

In some embodiments, each flat-ended collar is resiliently supported by a respective central post.

In some embodiments, each flat-ended collar is supported by a respective flange of a respective central post.

In some embodiments, each plurality of sharp probes is arranged in a regularly spaced array.

### BRIEF DESCRIPTION OF THE DRAWINGS

The invention will be more fully understood from the following detailed description, in conjunction with the following figures, wherein:

FIG. 1 is a top front right oblique view of an embodiment of a multiple allergy skin test device of the invention.

FIG. 2 is top view of the multiple allergy skin test device of FIG. 1.

FIG. 3 is a bottom front right oblique view of the multiple allergy skin test device of FIG. 1.

FIG. 4 is a bottom view of the multiple allergy skin test device of FIG. 1.

FIG. 5 is a side view of a test head of the multiple allergy skin test device of FIG 1.

FIG. 6 is an oblique bottom view of the test head of FIG. 5.

FIG. 7 is a close-up oblique bottom view of the test head of FIG. 6.

FIG. 8 is a rear left bottom oblique view of a portion of the multiple allergy skin test device of FIG. 1.

FIG. 9 is rear left bottom oblique view of the entire multiple allergy skin test device of FIG. 1.

FIG. 10 is another rear left bottom oblique view of the entire multiple allergy skin test device of FIG. 1.

FIG. 11 is rear view of the multiple allergy skin test device of FIG. 1.

FIG. 12 is a side view of a test head of the multiple allergy skin test device of FIG 1.

FIG. 13 is a top oblique view of a test head having three dull probes surrounding a plurality of sharp probes.

FIG. 14 is a bottom side oblique view of the test head of FIG. 13.

FIG. 15 is a top side oblique view of the test head of FIGS. 13 and 14.

FIG. 16 is a top side oblique view of a test head having a collar with a continuous flat dull edge.

FIG. 17 is a top side oblique view of the test head of FIG. 16.

FIG. 18 is a bottom side oblique view of the test head of FIGS. 16 and 17.

FIG. 19 is a top side oblique view of a test head having a collar with four raised dull nubs serving as dull probes, and seven square-base pyramids serving as sharp probes.

FIG. 20 is another top side oblique view of the test head of FIG. 19.

FIG. 21 is a bottom side oblique view of the test head of FIGS. 19 and 20.

FIG. 22 is a top side oblique view of a test head having eight slightly raised nubs serving as dull probes, and seven sharp cones serving as sharp probes.

FIG. 23 is another top side oblique view of the test head of FIG. 22.

FIG. 24 is a bottom side oblique view of the test head of FIGS. 22 and 23.

FIG. 25 is a top side oblique view of a test head having eight flattened nubs serving as dull probes, and seven sharp cones serving as sharp probes.

FIG. 26 is a top side oblique view of the test head of FIG. 25.

FIG. 27 is a bottom side oblique view of the test head of FIGS. 25 and 26.

FIG. 28 is a top side oblique view of a test head having five flattened and raised nubs supported by a broad collar, and seven sharp cones serving as sharp probes.

FIG. 29 is another top side oblique view of the test head of FIG. 28.

FIG. 30 is a bottom side oblique view of the test head of FIGS. 28 and 29.

FIG. 31 is a top side oblique view of a test head having a flange at the end of a central post, the flange supporting both five flattened and raised nubs, and seven sharp cones serving as sharp probes.

FIG. 32 is another top side oblique view of the test head of FIG. 31.

FIG. 33 is a bottom side oblique view of the test head of FIGS. 31 and 32.

FIG. 34 is a top side oblique view of a test head having a flange at the end of a central post supporting an integral collar having three dull contours serving as dull probes, and supporting seven sharp cones serving as sharp probes.

FIG. 35 is another top side oblique view of the test head of FIG. 34.

FIG. 36 is a bottom side oblique view of the test head of FIGS. 34 and 35.

FIG. 37 is a top side oblique view of a test head similar to FIGS. 1-6, for example, having five dull probes instead of six dull probes.

FIG. 38 is a side view of the test head of FIG. 37.

FIG. 39 is a is a bottom side oblique view of the test head of FIGS. 37 and 38.

FIG. 40 is a top side oblique view of an embodiment of a single tester with a single test head with a handle, having three dull probes supported by three respective arms, and seven square-base pyramids serving as sharp probes.

FIG. 41 is a bottom side oblique view of the single tester of FIG. 40.

FIG. 42 is a top view of the single tester of FIGS. 40 and 41.

FIG. 43 is a top side oblique view of an alternate test head that can have a handle as shown in FIGS. 40-42 so as to provide another embodiment of a single tester.

### DETAILED DESCRIPTION

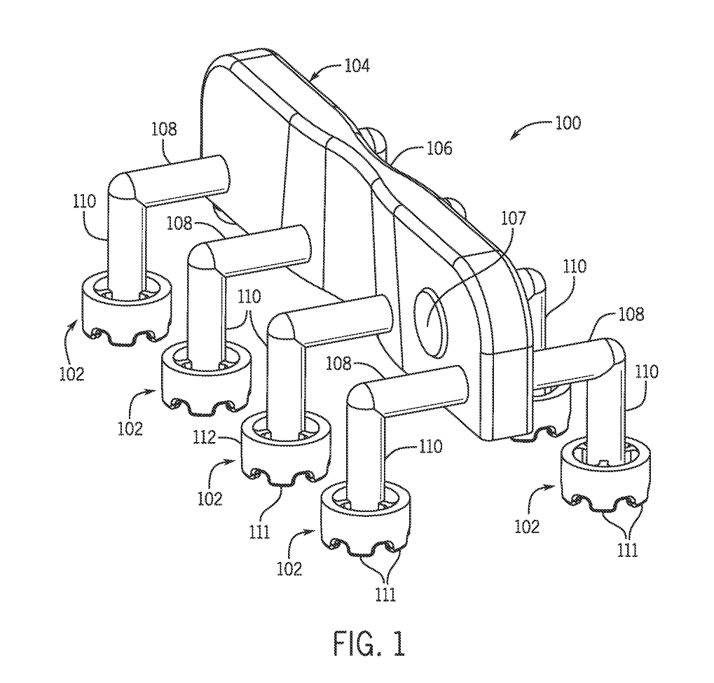

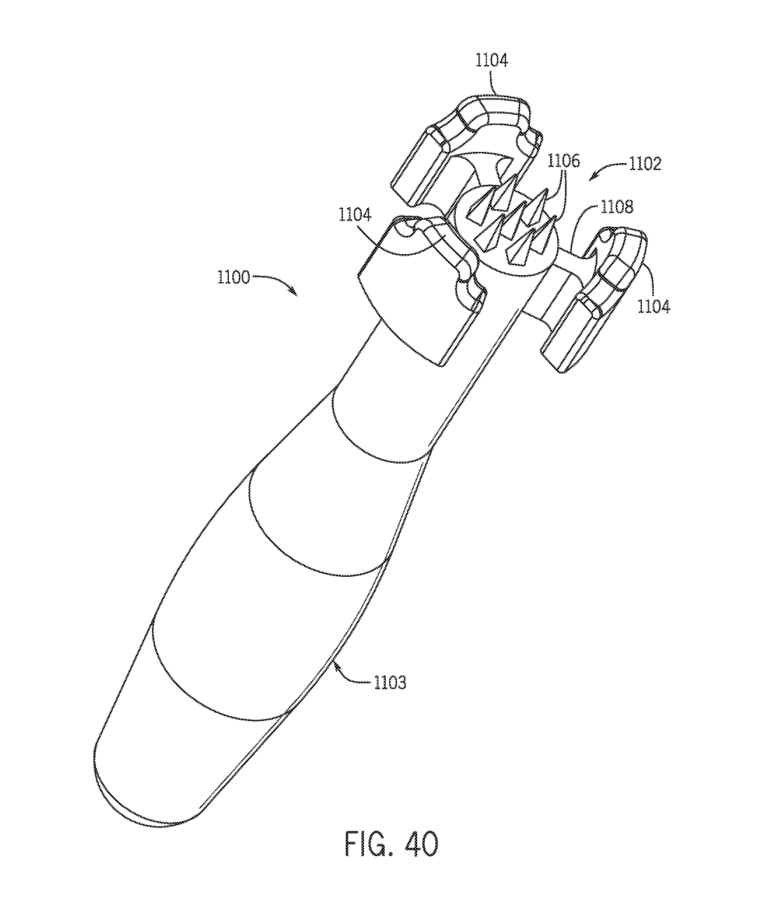

With reference to FIG. 1, a multiple allergy skin test device 100 of the invention is shown. This device 100 has eight test heads 102, for example. Other embodiments of such “multi-test” devices could have 2, 3, 4, 5, 6, 7, 9, or 10 test heads, for example. Or, a single test head projecting from the end of a suitable handle can be used (see, for example, FIG. 40).

The device 100 has a body 104, which has a gripping portion 106. The gripping portion is shown as a narrowing of the body 104. In other embodiments, the gripping portion can have a surface texture that enhances gripping, such as a stipple pattern, or a ridged pattern, for example. The device 100 also can also have an orientation indicator 107 on each side of the body 104, such as the raised circular areas 107. These raised circular areas 107 can resemble the eyes of a multi-legged animal, which can make the device more friendly for use with children.

The device also has one leg per test head 102, and so in this embodiment, there are eight legs 108, each of which supports one test head 102.

Each test head 102 has a central post 110 that extends from a respective leg 108. Each central post 110 terminates in a plurality of sharp probes (visible in later figures) for administering an allergen to skin. Alternatively, the central post of ANY embodiment described and/or claimed herein can have a single sharp probe. All discussion that mentions a plurality of sharp probes applies also to a singe sharp probe.

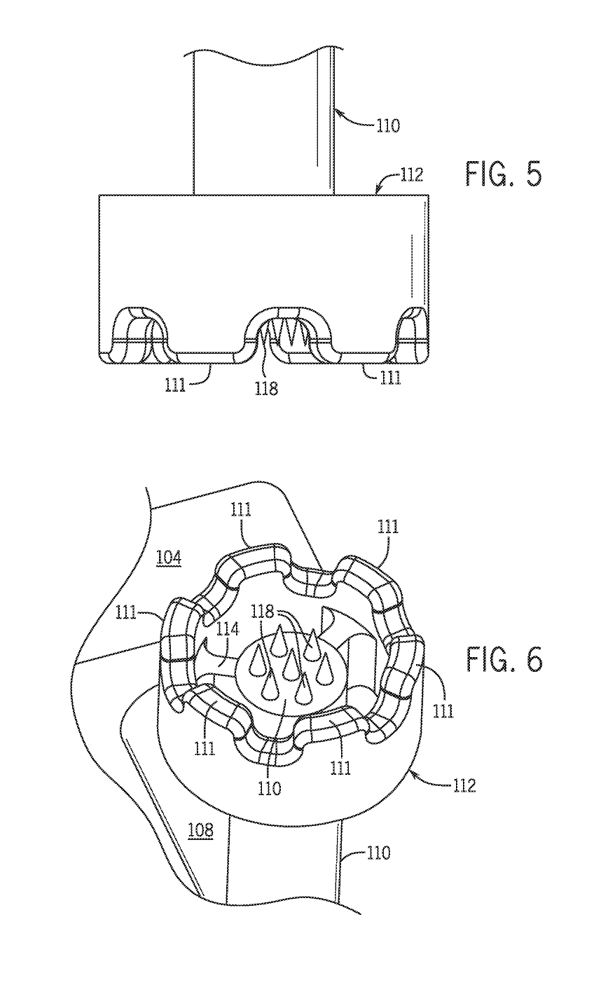

Each test head 102 also has a plurality of neighboring dull probes 111 that extend from the collar 112 of the test head 102, the dull probes 111 substantially surrounding the plurality of sharp probes (see for example, FIGS. 5, 6, 7). The dull probes 111 extend further in the direction of the central post than the sharp probes, such that the dull probes 111 contact the skin and apply perceivable pressure before the sharp probes contact the skin. After the sharp probes make contact with the skin, the sharp probes penetrate the skin, while the dull probes 111 press more firmly into the skin surrounding the skin penetrated by the sharp probes. The dull probes 111 thereby provide pressure that activates the “pain gate” effect, which blocks the painful sensations typically caused by the sharp probes entering the skin. Further, since each dull probe 111 activates a “pain gate”, a surrounding set of dull probes 111 effectively creates a “pain gate fence”, which reduces the pain due to the sharp probes better than a single dull probe surrounded by a plurality of sharp probes.

Likewise, a plurality of dull probes surrounded by a plurality of sharp probes reduces the pain created by the sharp probes penetrating the skin better than a single dull probe surrounded by a plurality of sharp probes, as taught by Heine et al.

An important aspect of the height difference is that the dull probes contact the skin before the sharp probes. It is important that the dull probes extend further than the sharp probes so that the dull probes apply perceivable pressure to the skin before any of the sharp probes first touch the skin. For example, the height of each dull probe can be about ⅛^{th} of an inch higher than the highest point of a respective sharp probe.

It is recommended that each test head be pressed squarely against the skin, i.e., pressed perpendicularly into the skin. One way to accomplish this is to cause all eight of the test heads to contact the skin at substantially the same time.

It's important to note that the sharp probes are NOT visible from most top and side views of the device 100, which is a very significant benefit in that children find the prospect of being stuck with sharp probes to be upsetting. Since the sharp probes are HIDDEN by the dull probes 111 of the collar 112 of the test head 102, the device 100 LOOKS much friendlier and less threatening than other known multi-test allergy skin testing devices having exposed sharp probes, such as U.S. Pat. No. 8,469,900 B2, for example. Because the device appears less threatening, the negative anticipation of a child before the device 100 is used on the skin of a child is MUCH less, which makes the reaction to any actual physical discomfort much less than if they child were emotionally upset. Thus, the non-threatening appearance of the device is synergistic with the reduced pain perception caused by the “pain gate” effect caused by the pressure of the dull probes 111 surrounding the sharp probes.

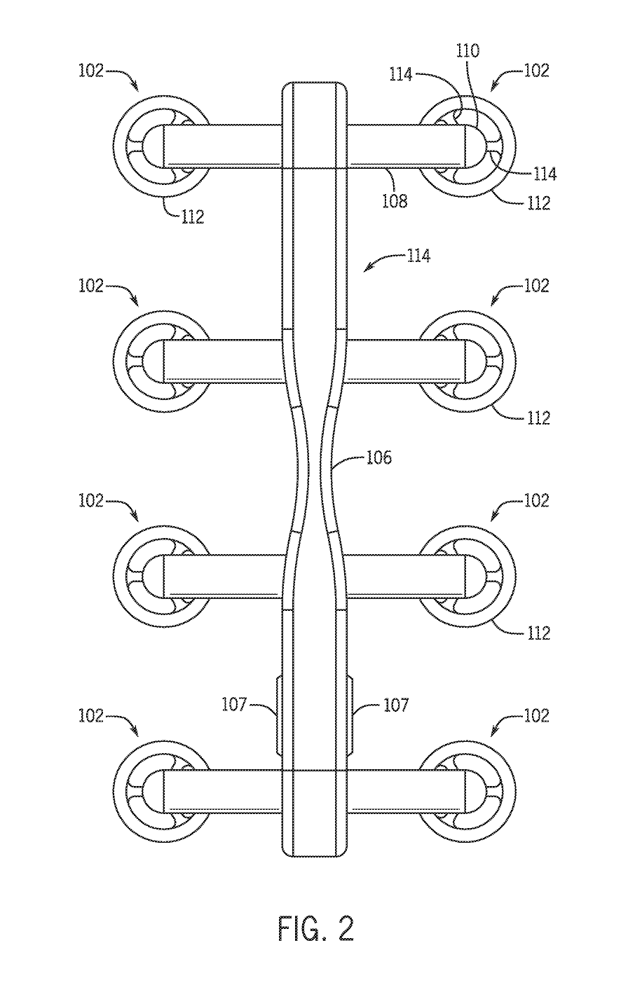

FIG. 2 shows a top view of the multiple allergy skin test device of FIG. 1, showing the body 104 having the gripping portion 106. An orientation indicator 107 is shown on each side of the body 104. The orientation indicators 107 help a user of the device 100 to correctly and consistently orient the device so as to create a consistent and understandable wheal pattern.

The legs 108 are shown, each supporting a test head 102. Each test head 102 includes a collar 112 and radial supports 114. The collar 112 is supported by the central post 110. In some embodiments, the supports 114 are substantially rigid. In other embodiments, the radial supports 114 have some resiliency.

Note that from this view, none of the sharp probes are visible.

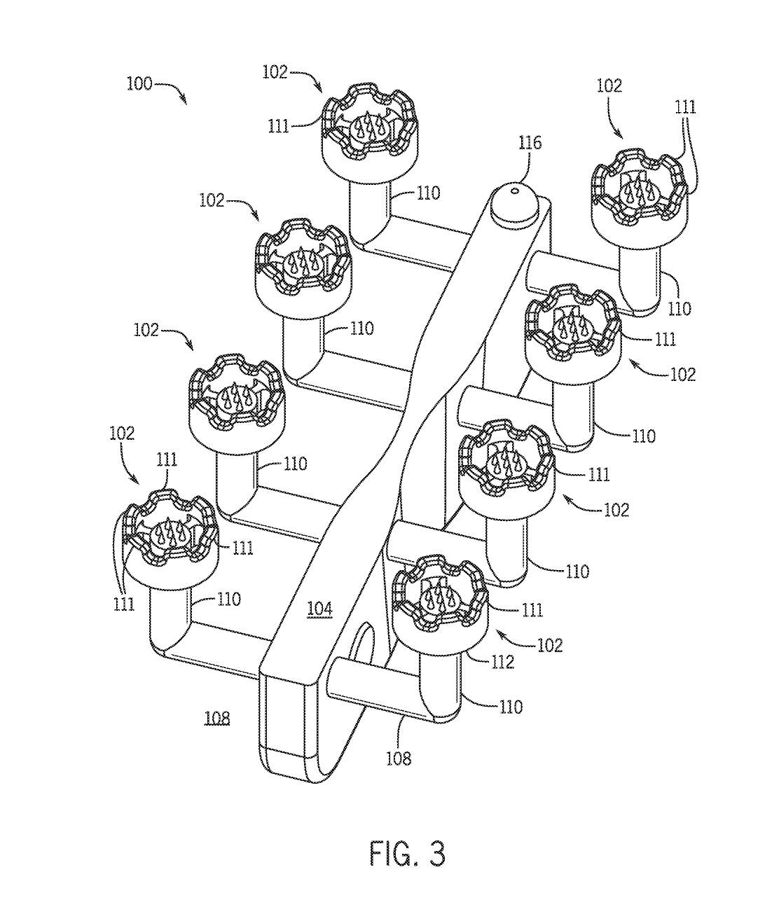

FIG. 3 shows the device 100 from the bottom, showing all eight test heads 102 from the bottom also. In this orientation, the sharp probes of each test head 102 are visible within the collar 112 and radial supports 114. In this view, there are seven sharp probes, and six dull probes 111 that extend from the collar 112 in the direction of the central post 110. Reference numbers for the sharp probes will be provided in subsequent drawings that provide a more close-up view. Note that the view of FIG. 3 can easily be hidden from a child, before, during, and after use of the device 100 on the skin of the child.

Also visible in FIG. 3 is the tray engagement extension 116 (extending from the bottom rear of the device 100) that can be used to disambiguate the orientation of the device 100 when inserted into an allergen tray (not shown). The allergen tray has a complementary indentation that receives the tray engagement extension 116 when the device 100 is correctly oriented in the tray. Without the tray engagement extension 116, the device 100 could be inserted into the tray in one of two possible orientations. However, since each test head can introduce a different allergen to the skin, it is important that the device 100 be insertable into the tray in only one orientation consistently. This enables a user to correctly map a particular allergen to a particular test head wheal in the skin of a patent being tested.

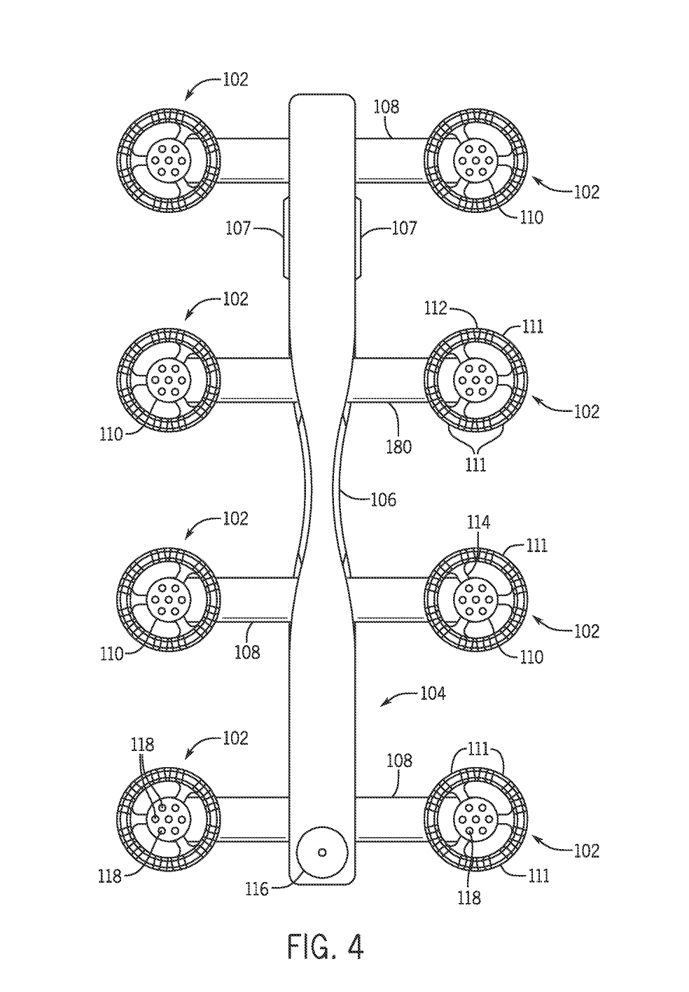

FIG. 4 is a bottom view of the multiple allergy skin test device of FIGS. 1, 2, and 3, showing eight test heads 102, each having six dull probes 111 that extend upward from the collar 112. Within the collar 112 are seven sharp probes 118 that extend upward from the central post 110.

FIG. 5 is a side view of a test head 102 of the multiple allergy skin test device 100 of FIGS. 1-4. This more close-up view shows the central post 110, the collar 112, and the dull probes 111 extending therefrom in the direction of the central post 110. Also extending from the central post 110 are the sharp probes 118, which are surrounded by the collar 112 and the dull probes 111.

FIG. 6 is an oblique bottom view of the test head of FIG. 5, showing the dull probes 111 projecting from the collar 112, that is supported by the radial supports 114. The dull probes 111 surround the sharp probes 118 that project from the central post 110. The central post 110 is supported by a leg 108 that extends outward from the body 104.

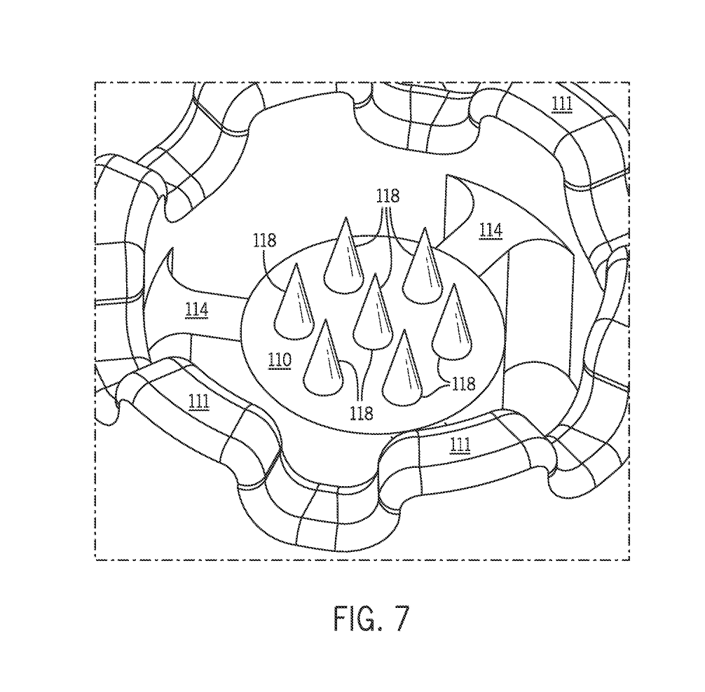

FIG. 7 is a close-up oblique bottom view of the test head of FIG. 6, showing the dull probes 111, that surround the sharp probes 118. The radial supports 114 are also shown extending radially outward from the central post 110 from which the sharp probes 118 project. In this embodiment, the sharp probes are cones, but they could be any shape that terminates in a sharp point, such as a pyramid with square base or a triangular base, for example.

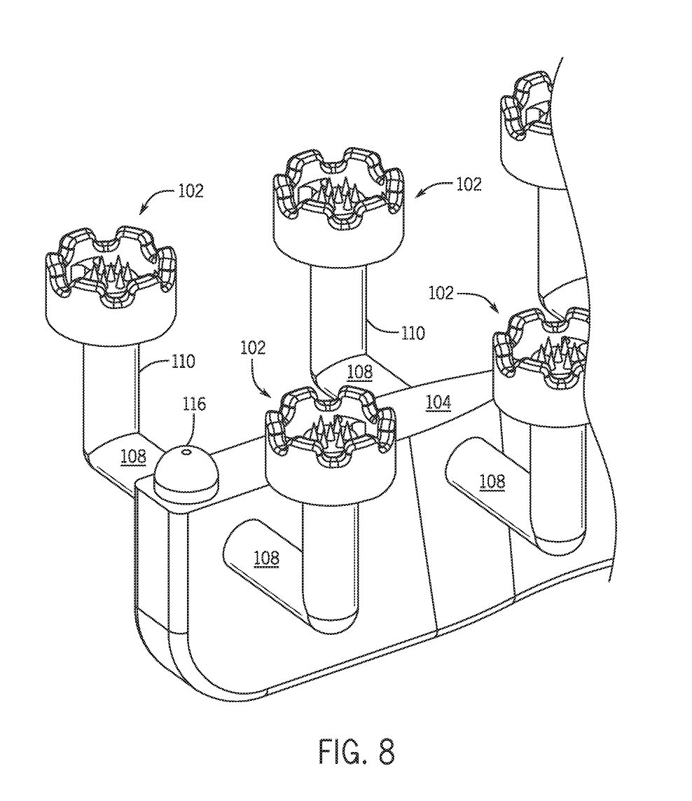

FIG. 8 is a rear left bottom oblique view of a portion of the multiple allergy skin test device of FIGS. 1-7, showing the body 104, four legs 108 extending therefrom, each leg supporting a central post 110 that terminates in a test head 102 (as shown in FIGS. 5-7). Also shown is a tray engagement extension 116 extending upward from the body 104.

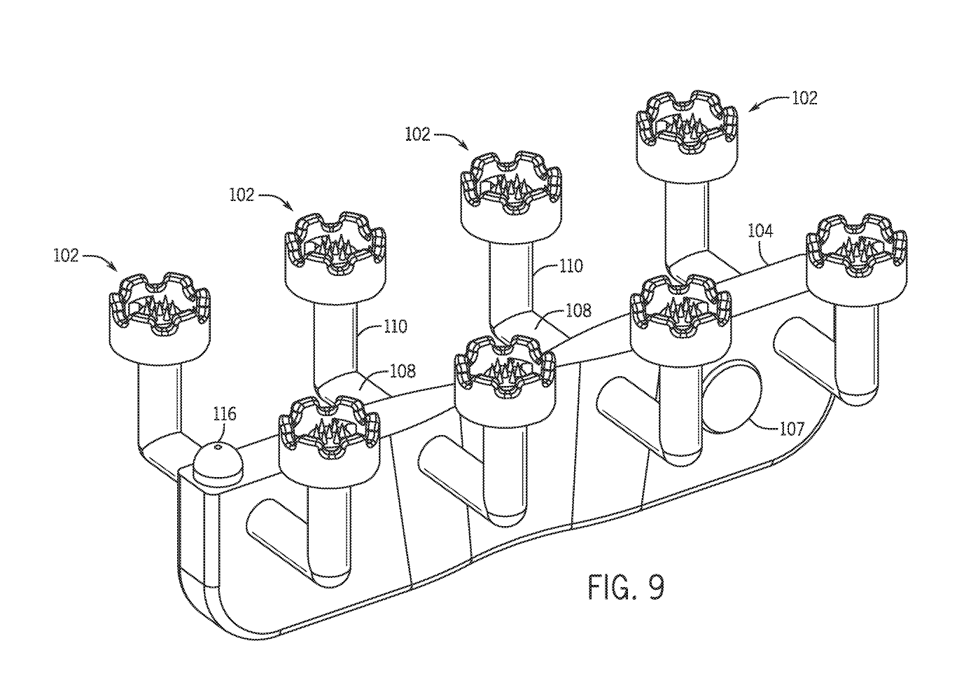

FIG. 9 is rear left bottom oblique view of the entire multiple allergy skin test device of FIGS. 1-8, showing the body 104, eight legs 108 extending therefrom, each leg supporting a central post 110 that terminates in a test head 102 (as shown in FIGS. 5-7). Also shown is the tray engagement extension 116 extending upward from the body 104, and the orientation indicator 107.

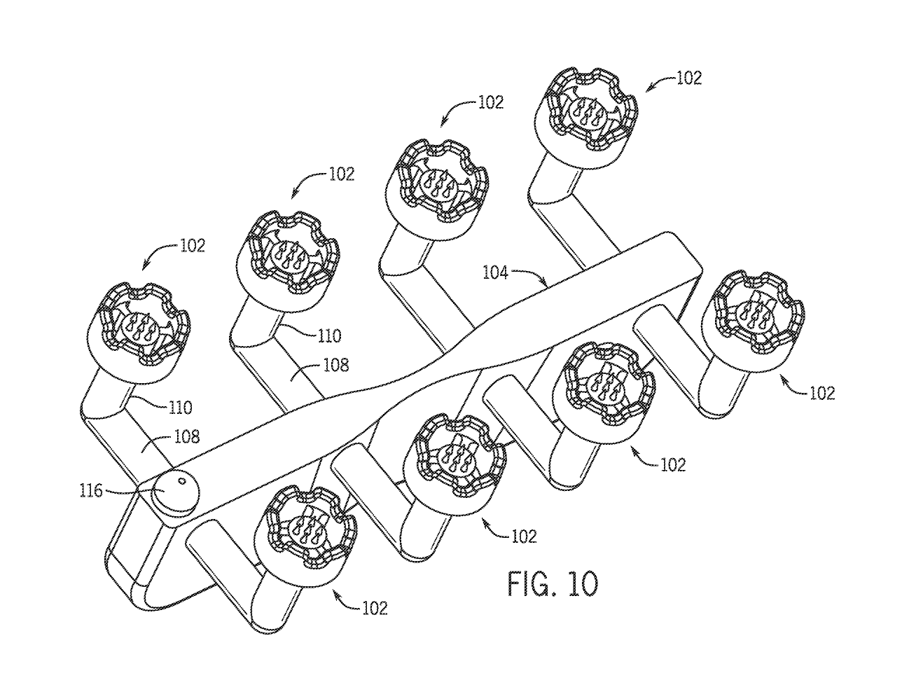

FIG. 10 is another rear left bottom oblique view of the entire multiple allergy skin test device of FIG. 1, showing eight test heads 102, supported by eight central posts 110, which are supported by eight legs 108, which are all supported by the body 104. The tray engagement extension 116 is seen extending upward from the body 104.

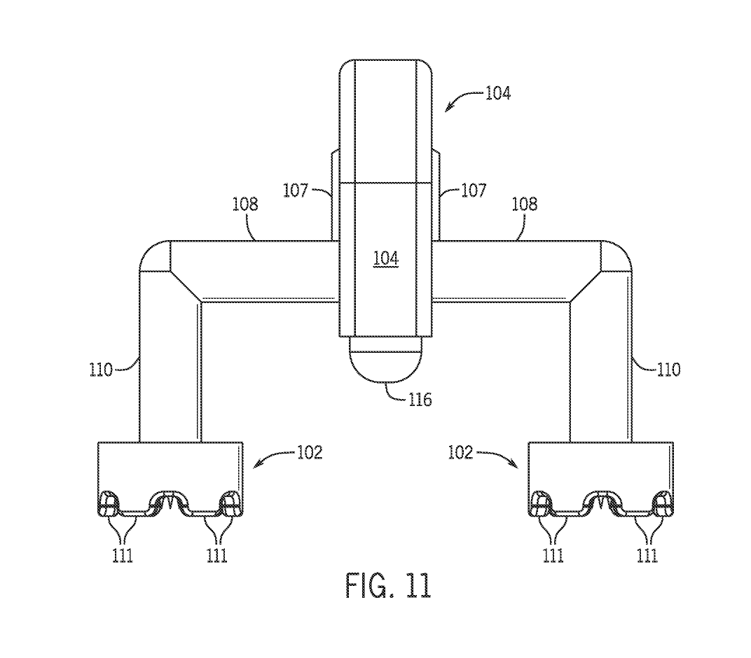

FIG. 11 is rear view of the multiple allergy skin test device of FIGS. 1-10, showing two test heads 102, each test head having six dull probes 111, four of which are visible in this figure, the other two being obscured by the two fully visible dull probes. Also shown is the body 104, two legs 108 extending outward therefrom, each leg supporting a central post 110 that terminates in a test head 102 (as shown in FIGS. 5-7). Also shown is the tray engagement extension 116 extending downward from the body 104, and the orientation indicator 107 on each side of the body 104.

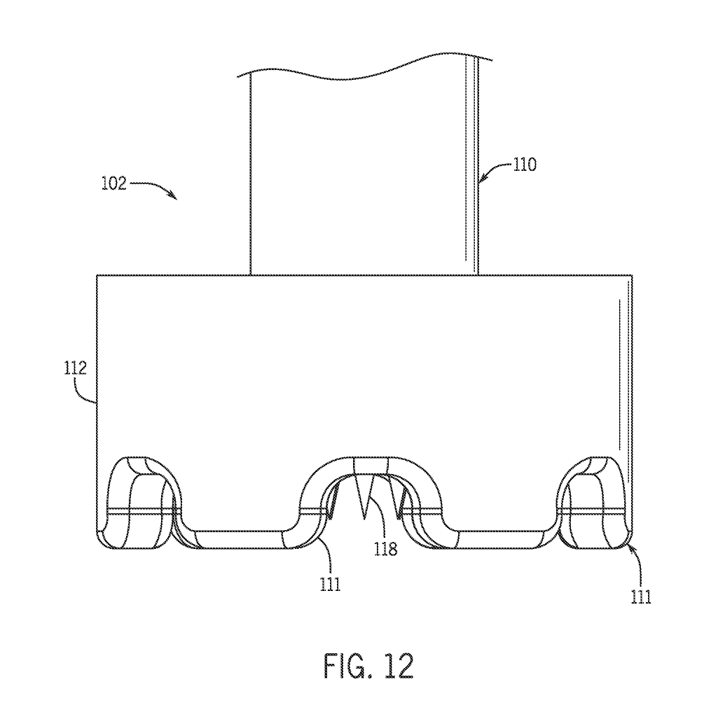

FIG. 12 is a side view of a test head 102 of the multiple allergy skin test device 100 of FIGS. 1-11. A single test head 102 is shown having a central post 110 that terminates in a plurality of sharp probes 118 that are surrounded by a plurality of dull probes 111, here showing four dull probes 111 that project from the collar 112.

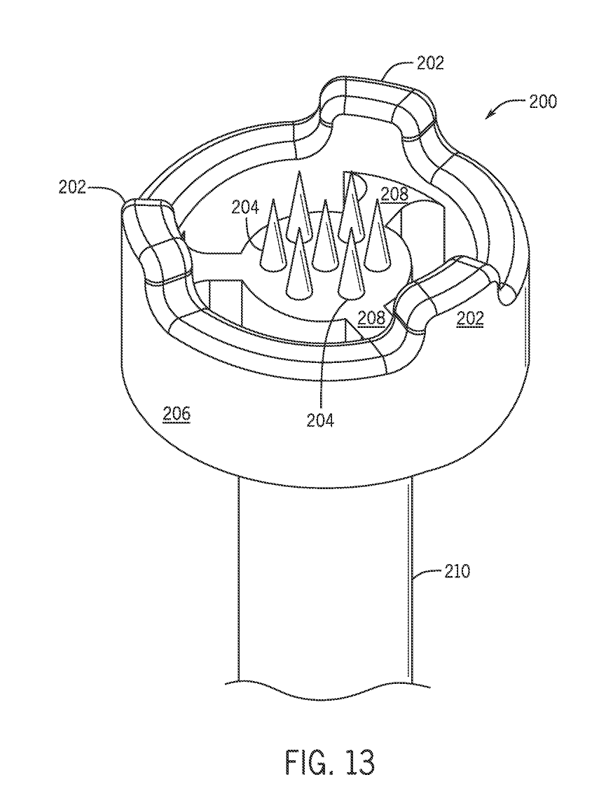

FIG. 13 is a top oblique view of a test head 200 having three dull probes 202 surrounding a plurality of seven sharp probes 204. Alternatively, there could be three, four, five, six or eight sharp probes 204, for example. The dull probes 202 in this embodiment are contours of a collar 206 that is supported by three radial supports 208 that are supported by a central post 210. In some embodiments, there can be two or four radial supports, for example.

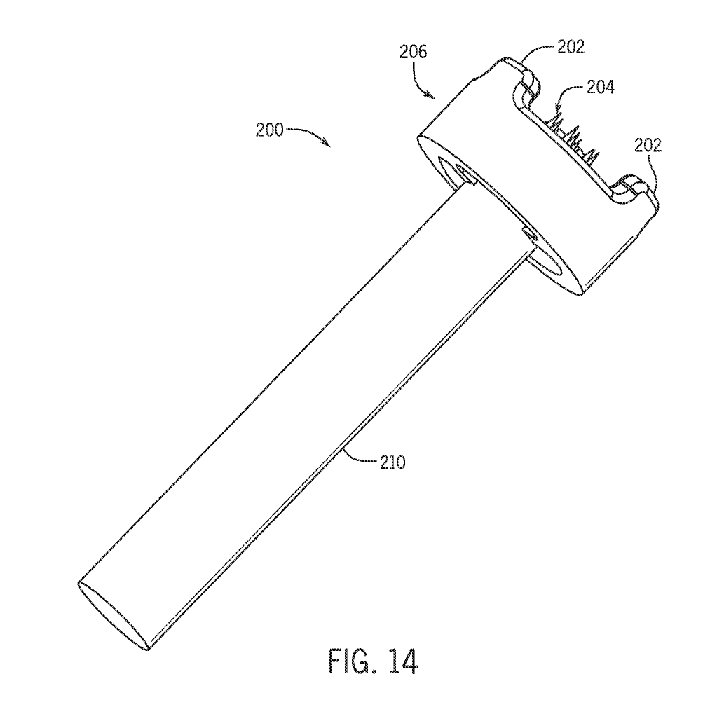

FIG. 14 is a bottom side oblique view of the test head 200 of FIG. 13. It is evident from this view that the sharp probes 204 are barely visible, the sharp probes 204 being substantially hidden by the collar 206, and so will not provoke anxiety in a child to be tested.

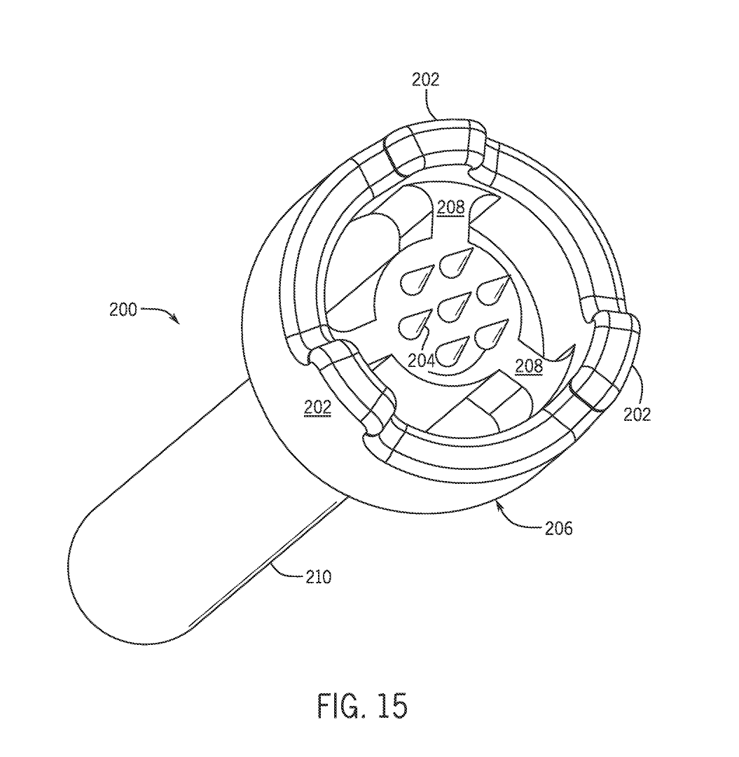

FIG. 15 is a top side oblique view of the test head 200 of FIGS. 13 and 14. The dull probes 202 in this embodiment are contours of the collar 206 that is supported by three radial supports 208 that are supported by a central post 210. In this embodiment, the sharp probes 204 are sharp cones, but they could also be other sharp shapes, such as a triangular-base pyramid, an oval sharp cone, or a hexagonal-base pyramid, for example.

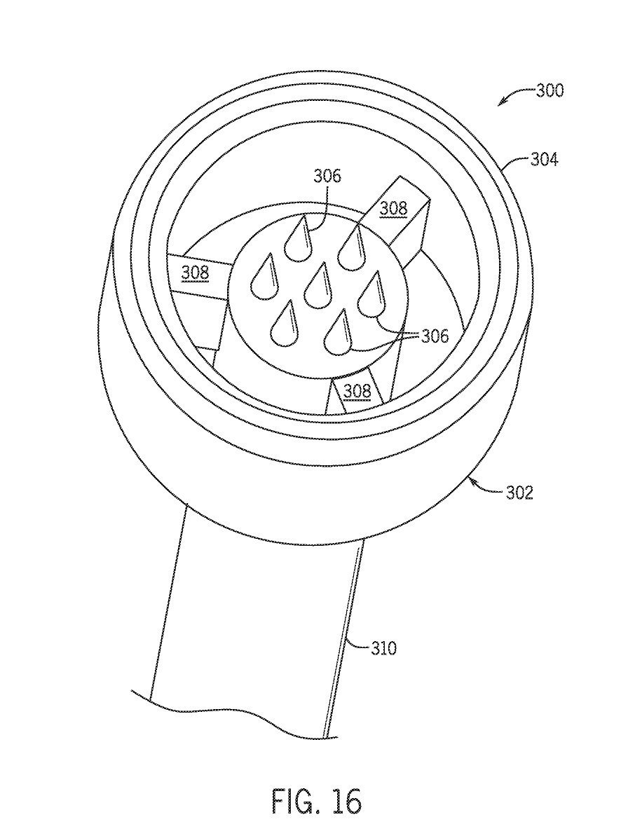

FIG. 16 is a top side oblique view of a test head 300 having a collar 302 with a continuous flat dull edge 304. The continuous flat dull edge 304 serves as a continuous dull probe that presses against the skin before the sharp probes 306 penetrate the skin. The pressure of the dull edge 304 surrounding the sharp probes 306 creates a surrounding pain gate effect termed herein as a “pain-gate fence”, also referred to as PAIN FENCE™ technology, i.e., a spatially distributed, at least partially surrounding dull pressure that fences in the pain of the sharp probes 306 so that the pain cannot escape to reach the brain, and thereby cannot be felt. The collar 302 is supported by three radial supports 308 that are supported by a central post 310.

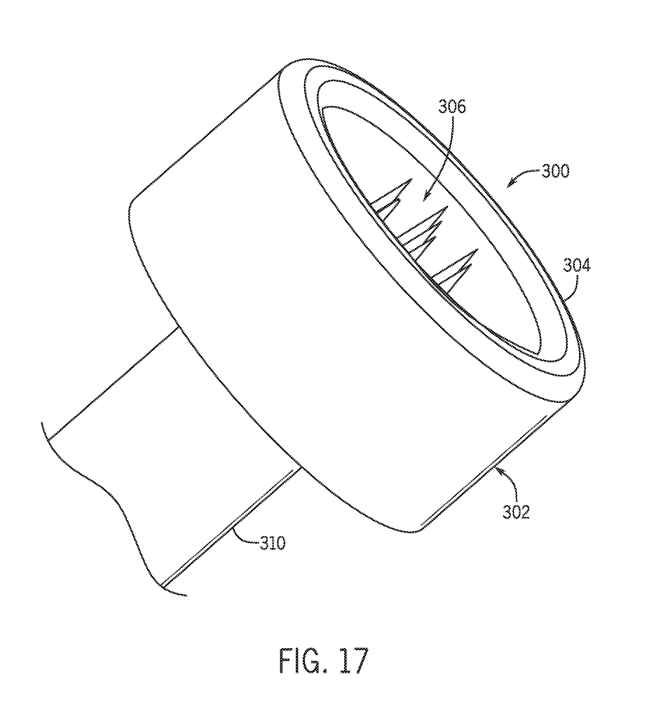

FIG. 17 is a top side oblique view of the test head 300 of FIG. 16. Notice that the collar 302 substantially hides the sharp probes 306 from view. The continuous flat dull edge 304 surrounds the sharp probes 306, contacting the skin before the sharp probes 306 contact the skin. The sharp probes 306 extend from the central post 310.

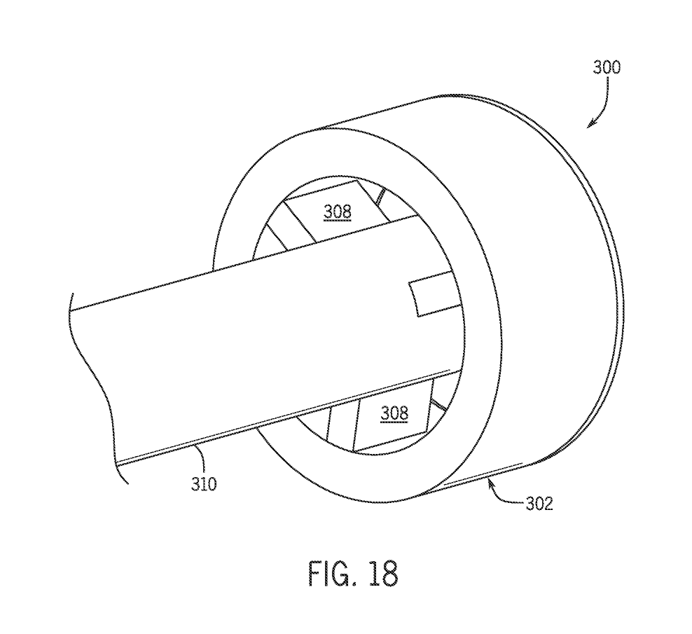

FIG. 18 is a bottom side oblique view of the test head 300 of FIGS. 16 and 17, showing the three radial supports 308 that support the collar 302 upon the central post 310.

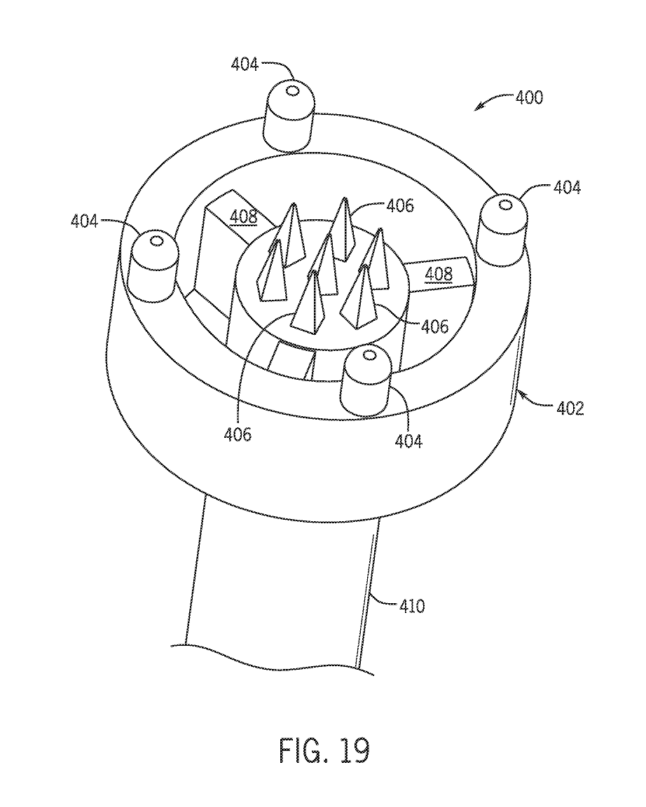

FIG. 19 is a top side oblique view of a test head 400 having a collar 402 with four raised dull nubs serving as dull probes 404, and seven square-base pyramids serving as sharp probes 406. The collar 402 is supported by the three radial supports 408 that are supported by the central post 410. Alternatively, there could be only six sharp probes 407, and six dull probes 404, one dull probe per sharp probe. In this alternate embodiment, the sharp probes 404 could be arranged equidistantly about the periphery of the round end of the central post 410, and the six dull probes 404 could be disposed on the collar 402 either matched to or located between each sharp probe 406.

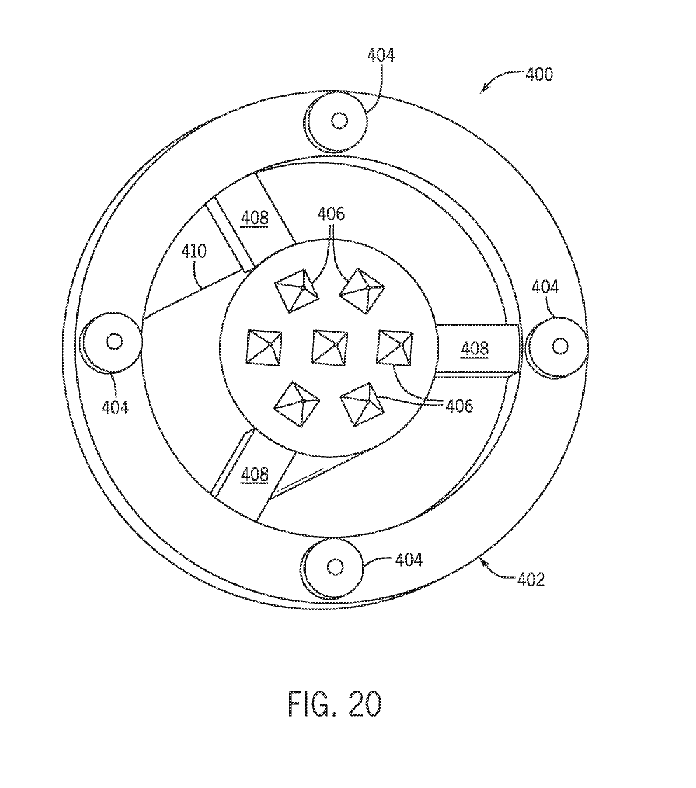

FIG. 20 is another top side oblique view of the test head 400 of FIG. 19, showing the central post 410 supporting the seven sharp square-base pyramid probes 406. FIG. 20 also shows the four dull probes 404 that are supported by the collar 402, which is supported by the three radial supports 408, that are supported by the central post 410.

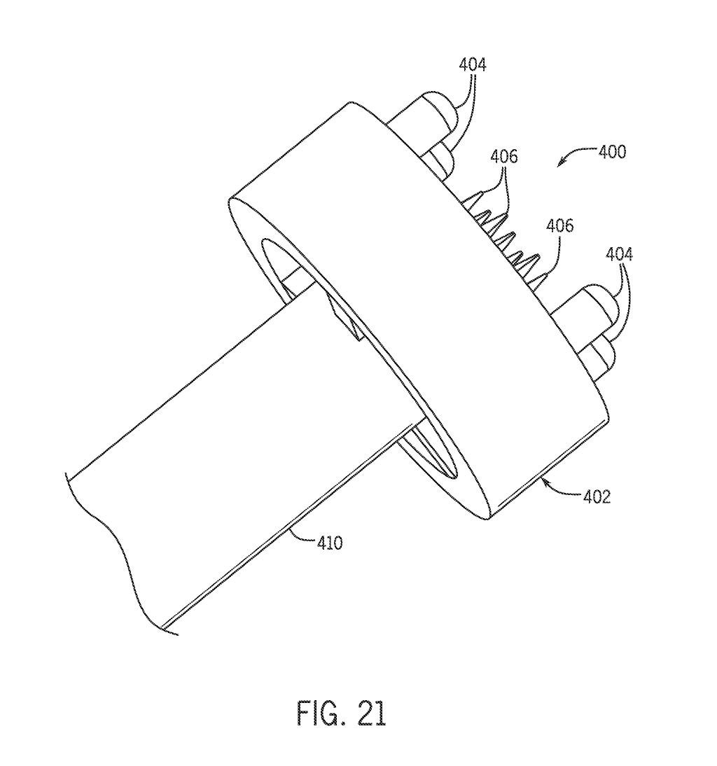

FIG. 21 is a bottom side oblique view of the test head 400 of FIGS. 19 and 20, showing the central post 410 supporting the collar 402 that supports the four dull probes 404, as well as the seven sharp probes 406. Note that in this view it can be seen that the dull probes 404 extend further along the direction of the central post 410 than the sharp probes 406, ensuring that the dull probes 404 will contact the skin before the sharp probes 406 contact the skin.

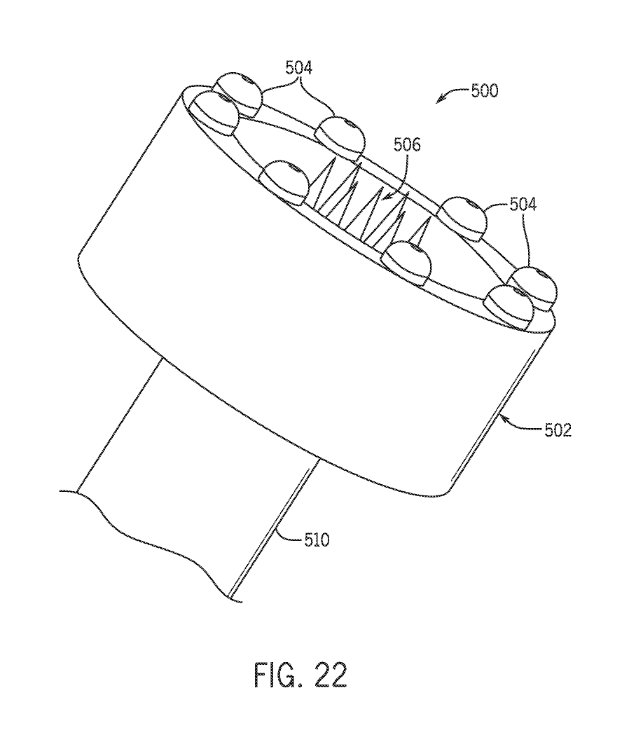

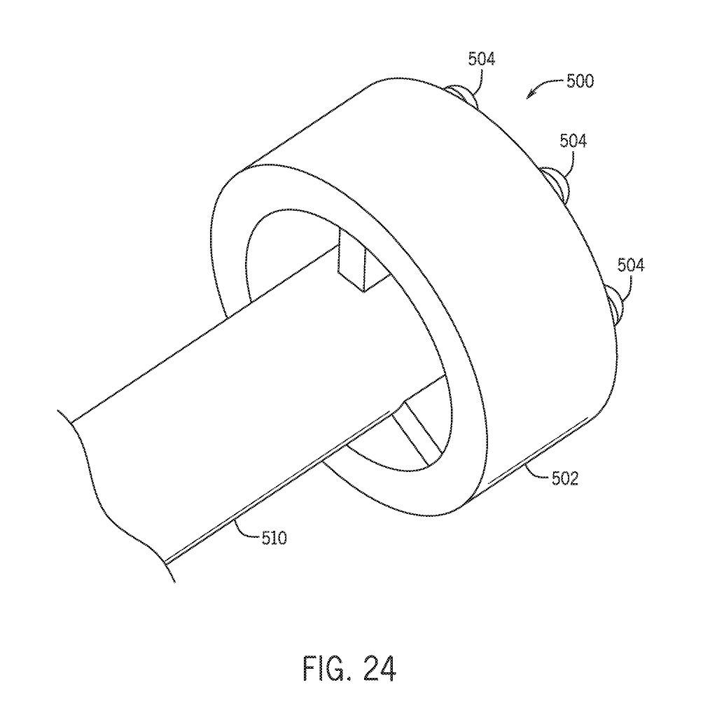

FIG. 22 is a top side oblique view of a test head 500 having eight slightly raised nubs serving as dull probes 504, and seven sharp cones serving as sharp probes 506. The dull probes 504 are supported on the collar 502, and the collar 502 is supported by the radial supports 508 (not shown in this figure), which are supported by the central post 510. There can be two, three, four, or five radial supports 508, for example. There can be three, four, five, six, seven, eight, or nine sharp probes 506, for example. There can be three, four, five, six, seven, eight, nine, or ten dull probes 504, for example. Notice that most of the sharp probes 506 are hidden from view via most angles, especially from bottom side oblique views, as shown in FIG. 24.

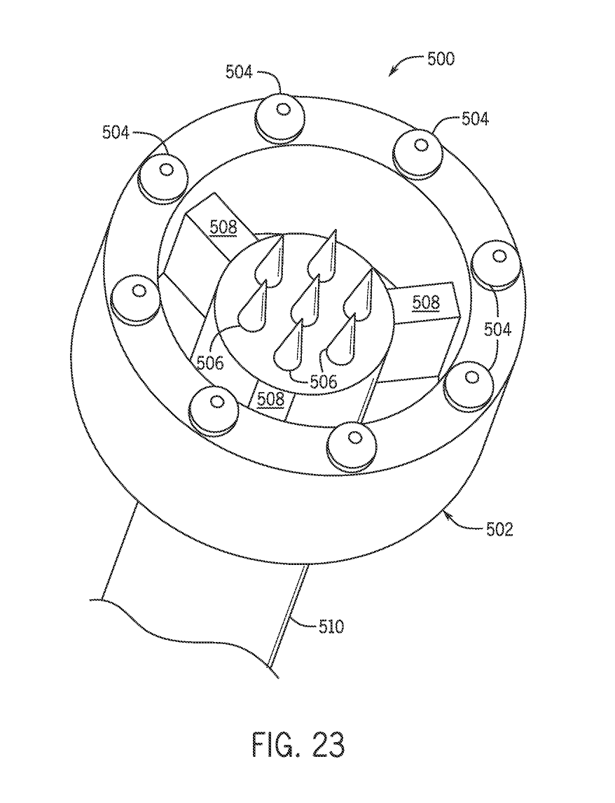

FIG. 23 is another top side oblique view of the test head of FIG. 22, showing the collar 502 supporting the surrounding dull probes 504 that provide a pain-gate fence to block pain sensations caused by the plurality of sharp probes 506 when they penetrate the skin of a person to be allergy skin tested. The collar 502 is supported by the radial supports 508 that are attached to the central post 510.

FIG. 24 is a bottom side oblique view of the test head 500 of FIGS. 22 and 23. In this view, no sharp probes are visible, and most of the dull probes 504 are hidden as well by the collar 502 that is pressed into the skin by movement of the central post 510 towards the skin.

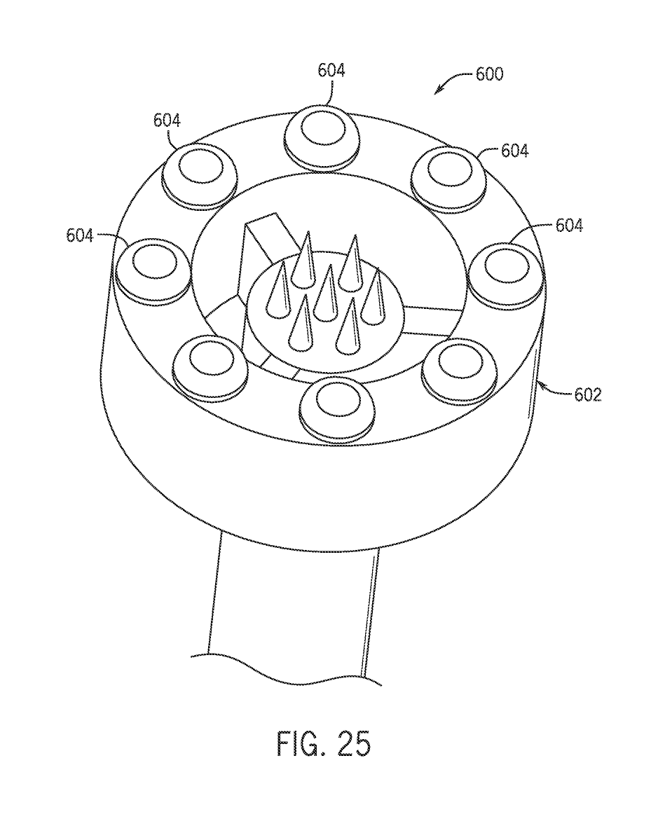

FIG. 25 is a top side oblique view of a test head 600 having a collar 602 that is wider than the collar 502 of FIGS. 22-24, the collar 502 having eight flattened nubs serving as dull probes 604 that are wider and flatter than the dull probes 504 of FIGS. 22-24. In other embodiments, there could be three, four, five, six, seven, or nine dull probes, for example.

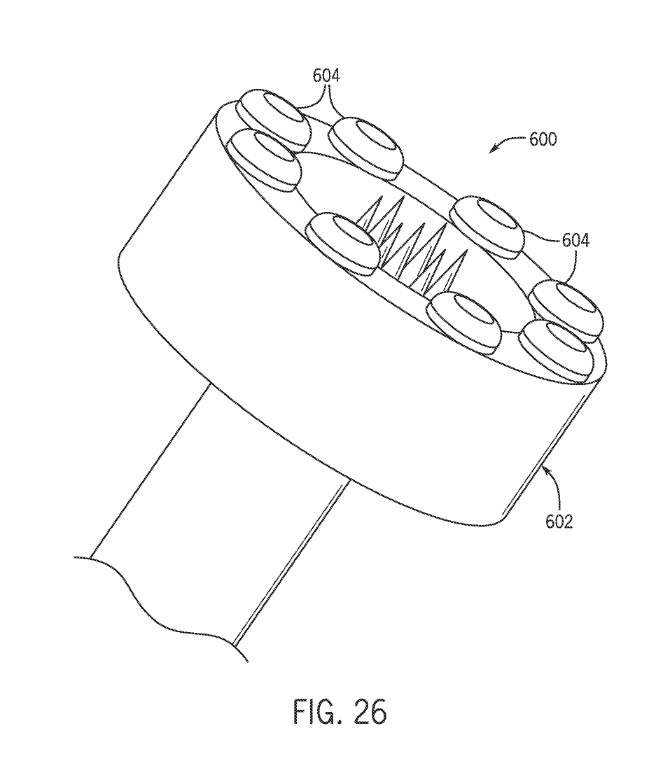

FIG. 26 is a top side oblique view of the test head 600 of FIG. 25, showing the eight dull probes 604 supported by the surrounding collar 602.

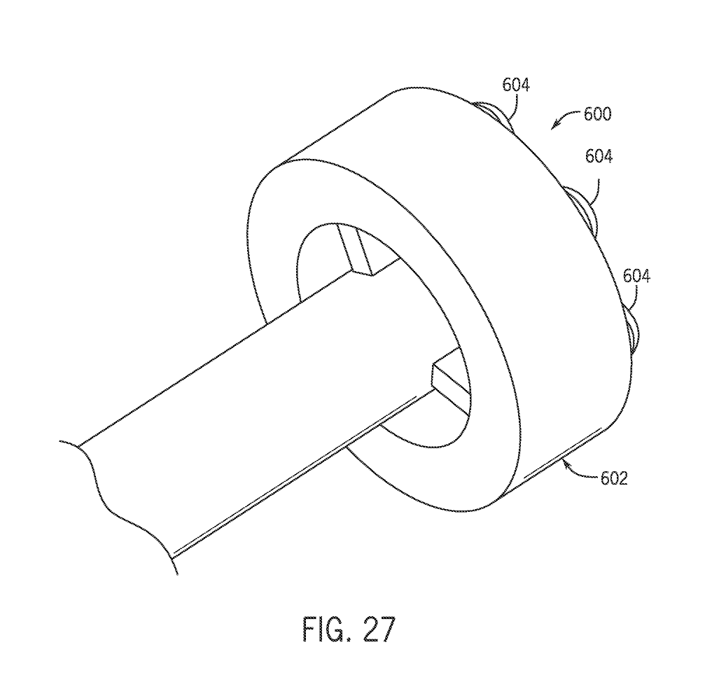

FIG. 27 is a bottom side oblique view of the test head 600 of FIGS. 25 and 26 FIGS. 22-24, showing the collar 602 supporting the dull probes 604, this view totally hiding all the sharp probes of the test head 600.

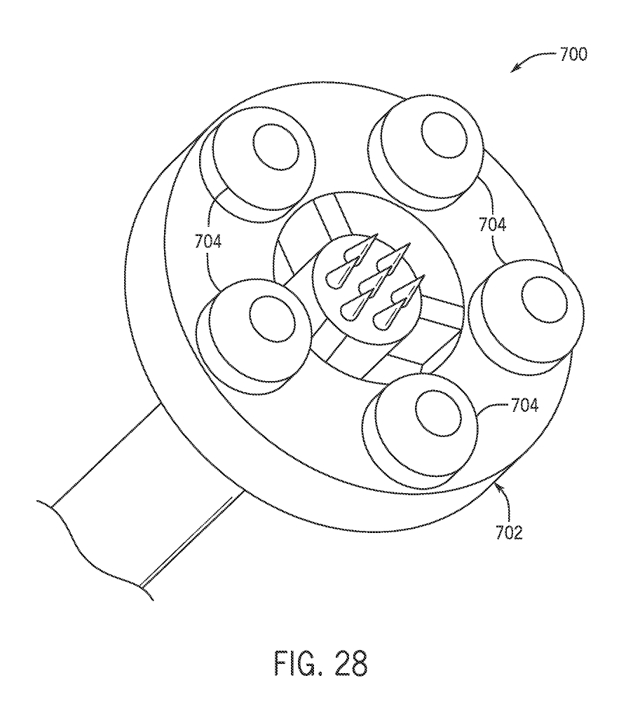

FIG. 28 is a top side oblique view of a test head 700 having five flattened and raised nubs serving as dull probes 704 that are supported by a collar 702 that is broader and flatter than the collar 602 of FIGS. 25-27.

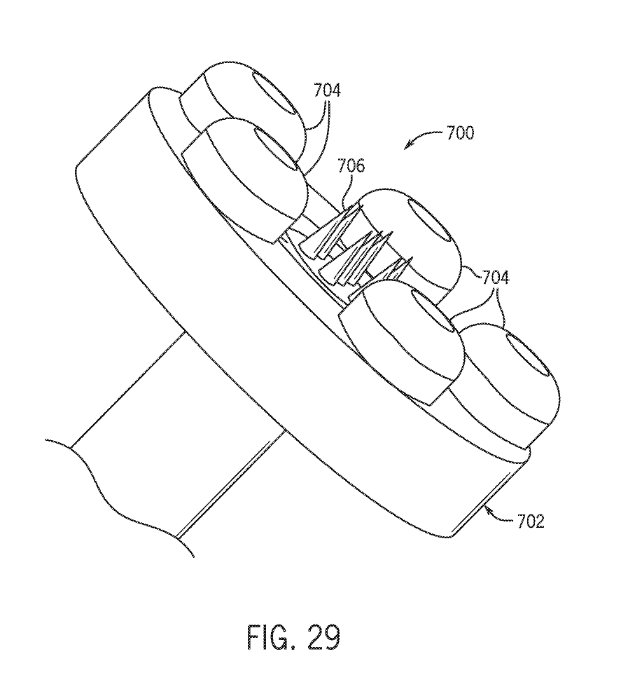

FIG. 29 is another top side oblique view of the test head 700 of FIG. 28, showing how the dull probes 704 can at least partially hide the sharp probes 706. In other embodiments, there could be three, four, or six dull probes 704.

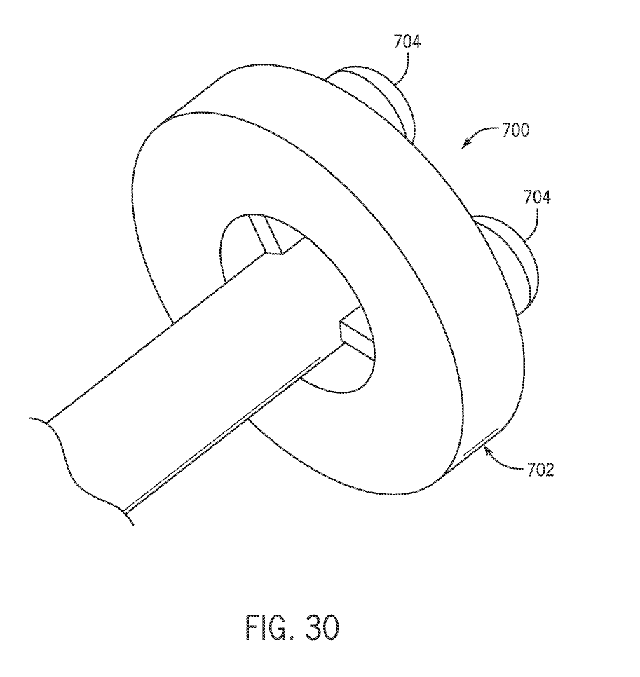

FIG. 30 is a bottom side oblique view of the test head 700 of FIGS. 28 and 29, showing how the collar 702 hides all sharp probes from view when seen from bottom perspectives.

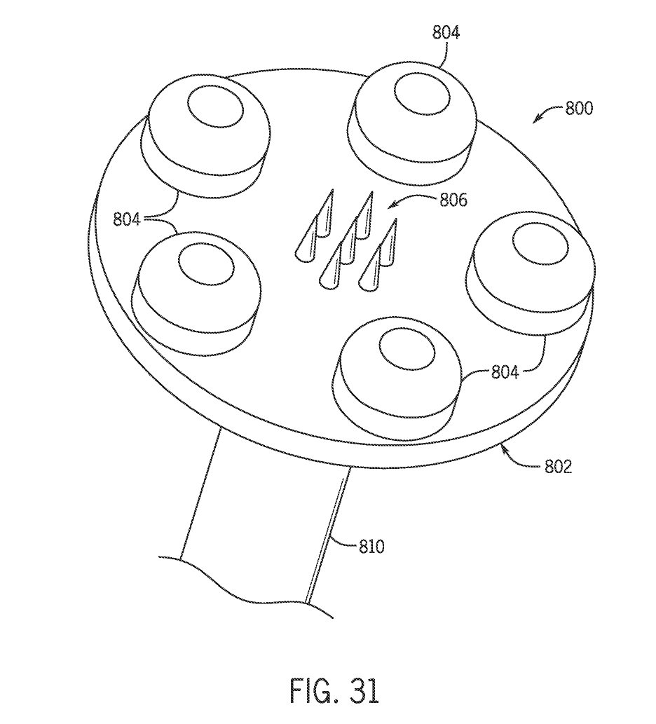

FIG. 31 is a top side oblique view of a test head 800 having a flange 802 at the end of a central post 810, the flange 802 supporting both five flattened and raised nubs that serve as dull probes 804, and seven sharp cones serving as sharp probes 806.

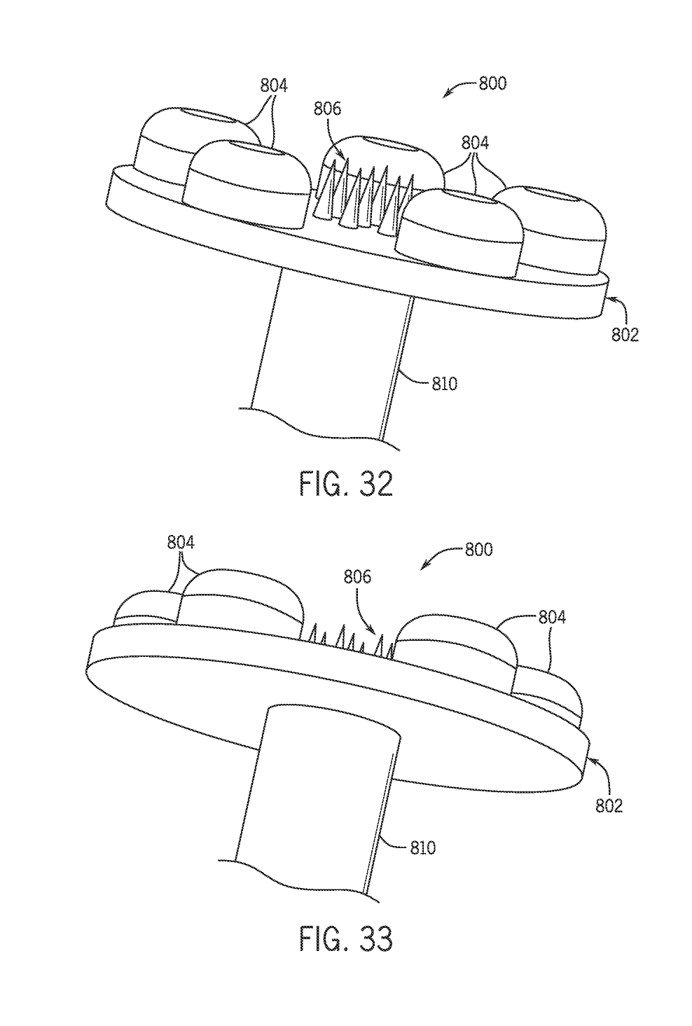

FIG. 32 is another top side oblique view of the test head of FIG. 31. In this view of this embodiment, the dull probes 804 can be seen to be taller than the thickness of the flange 802. In other embodiments, there can be three, four, or six dull probes 804 supported by the flange 802 that is extending from the central post 810. These dull probes all surround the sharp probes 806.

FIG. 33 is a bottom side oblique view of the test head 800 of FIGS. 31 and 32, showing how the flange 802 serves to at least partially hide the sharp probes 806. When the central post 810 is pressed towards the skin of a patient, the dull probes 804 contact the skin first, and then the sharp probes 806 penetrate the skin to deliver the allergen to be tested.

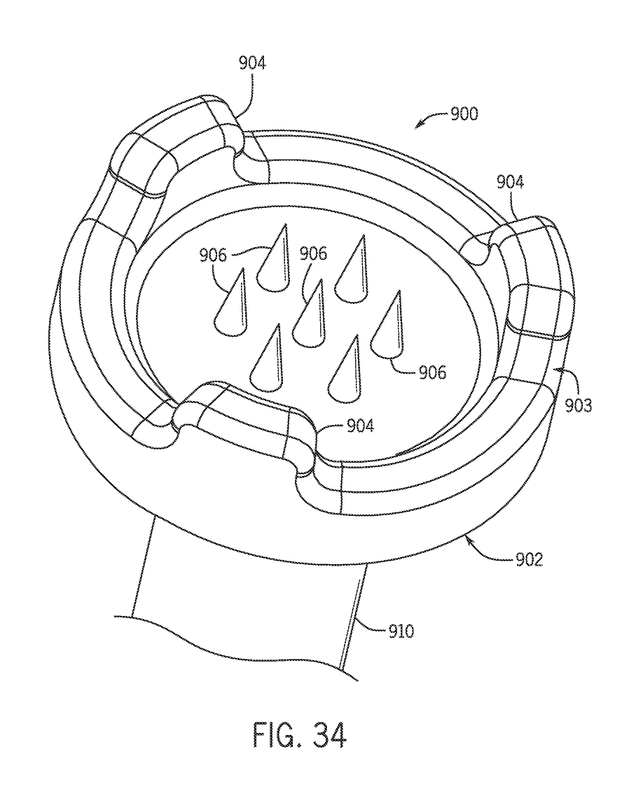

FIG. 34 is a top side oblique view of a test head 900 having a flange 902 at the end of a central post 910 that supports an integral collar 903 with three dull contours serving as dull probes 904, and supporting seven sharp cones serving as sharp probes 906. The integral collar 903 rises above the flange 902, and can have three, four, five, six, or seven dull contours to serve as dull probes 904. The dull probes 904 rise higher than the sharp probes 906, such that the dull probes 904 are pressed into the skin of a person to be tested before the sharp probes 906 penetrate into the skin.

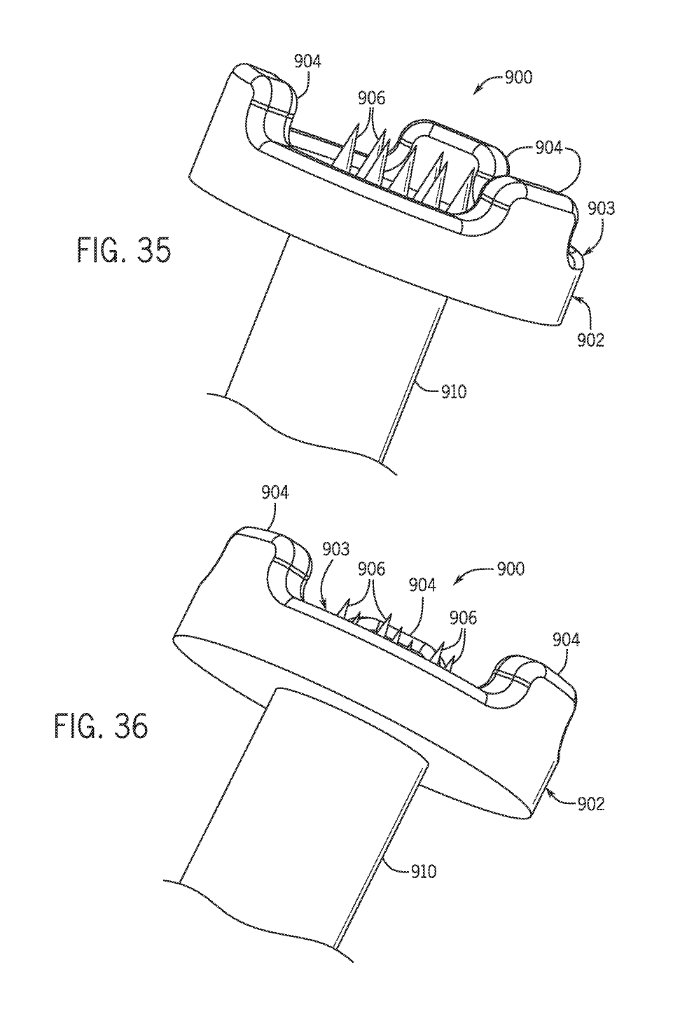

FIG. 35 is another top side oblique view of the test head 900 of FIG. 34, showing that the dull probes 904 are taller than the sharp probes 906, so that the dull probes 904 are pressed into the skin before the sharp probes 906, so that a pain gate effect is initiated before the sharp probes 906 contact the skin of the patient.

FIG. 36 is a bottom side oblique view of the test head 900 of FIGS. 34 and 35, showing that the collar 903 and the flange 902, and the dull probes 904 all cooperate to substantially hide the sharp probes 906 from view as the test head 900 is pressed downward towards the skin of the patient.

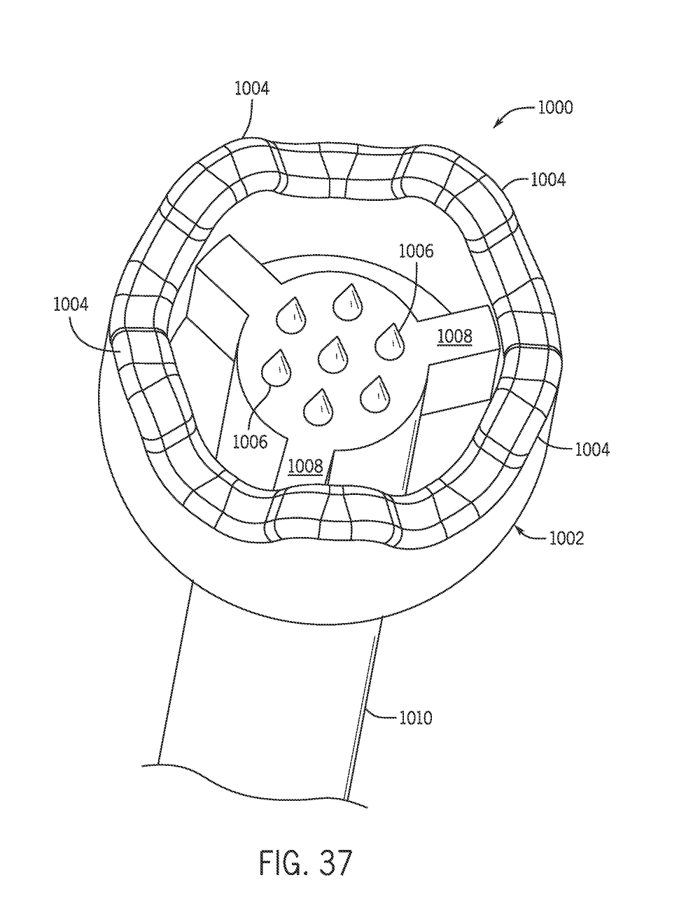

FIG. 37 is a top side oblique view of a test head 1000 similar to FIGS. 1-6, for example, having five dull probes 1004 instead of six dull probes, shaped as slightly flattened sinusoidal undulations in the dull edge of the collar 1002. The collar 1002 and dull probes 1004 surround the sharp probes 1006 supported by the central post 1010 that also supports the radial suports 1008 that support the collar 1002.

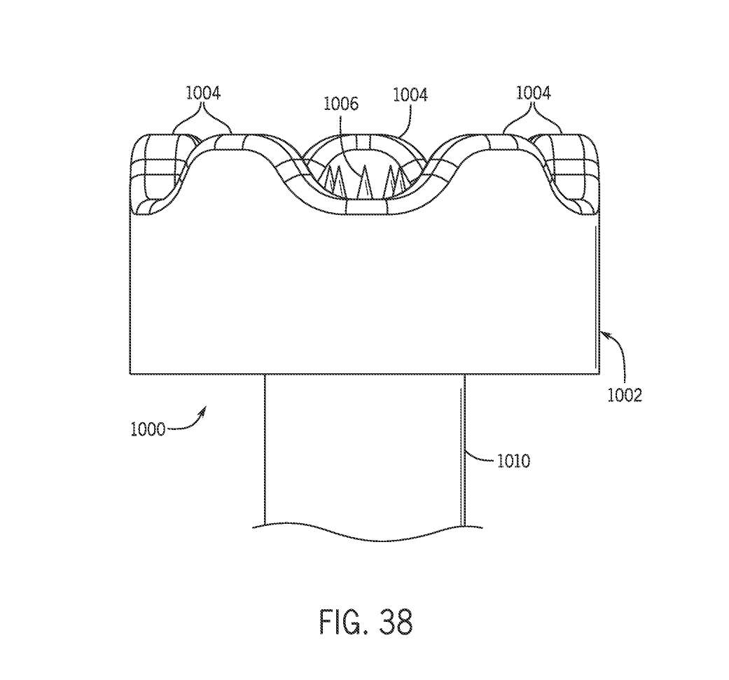

FIG. 38 is a side view of the test head 1000 of FIG. 37, showing how the collar 1002 and the undulations that serve as dull probes 1004 substantially hide the sharp probes 1006 from view from the side.

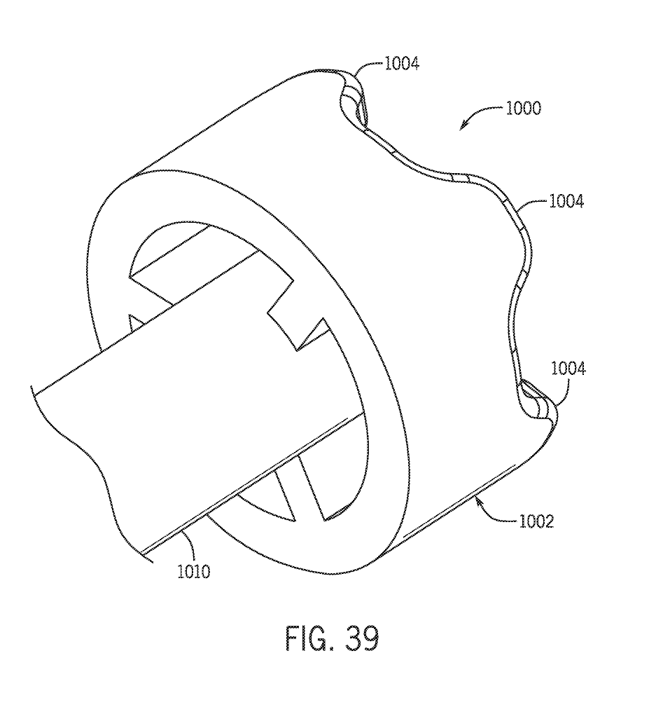

FIG. 39 is a is a bottom side oblique view of the test head 1000 of FIGS. 37 and 38. Showing how the sharp probes 1006 are totally hidden.

FIG. 40 is a top side oblique view of a single tester device 1100 with a single test head 1102 controlled by a handle 1103, the single test head 1101 having three dull probes 1104 supported by three respective radial supports 1108, and seven square-base pyramids serving as sharp probes 1106. This single device 1100 is for testing a single allergen, whereas the device shown in FIG. 1, for example, is for testing multiple allergens, one allergen per test head 102. In other embodiments, the test head 1101 can have only a single sharp probe.

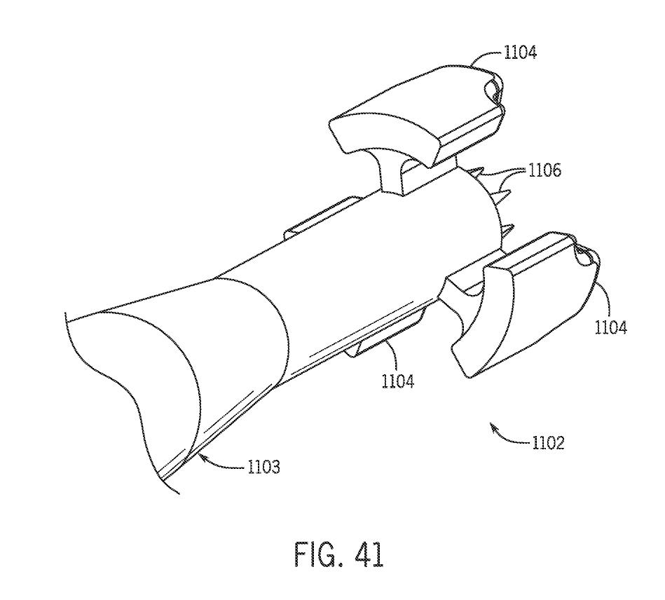

FIG. 41 is a bottom side oblique view of the single tester device 1100 of FIG. 40.

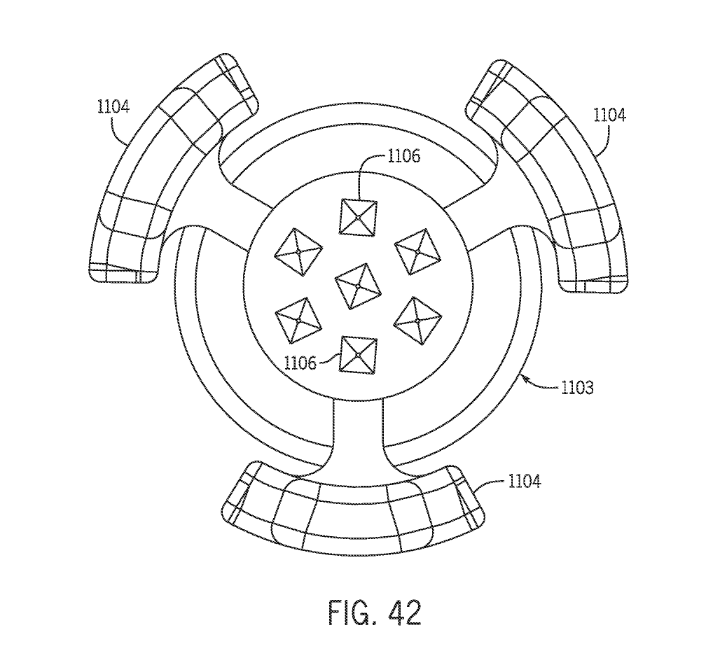

FIG. 42 is a top view of the single tester device of FIGS. 40 and 41, showing the dull proes 1104, the sharp probes 1106, and the handle 1103.

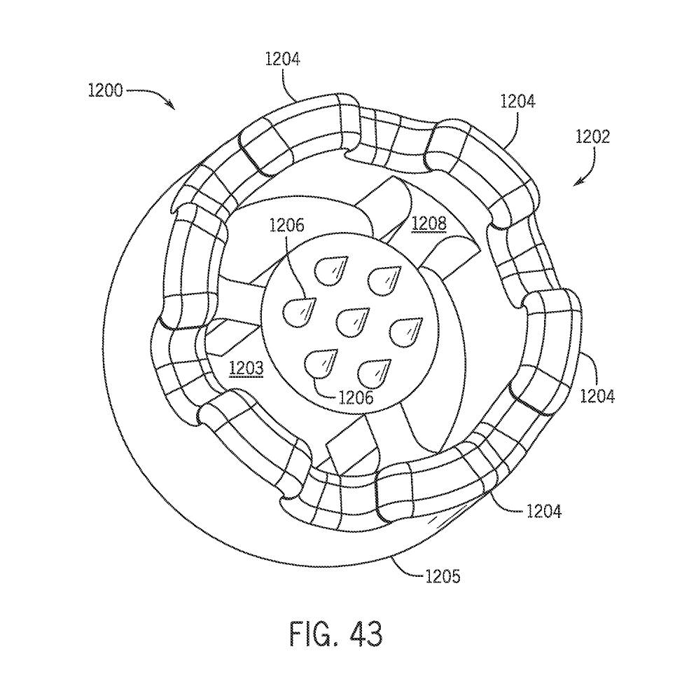

FIG. 43 is a top side oblique view of an alternate test head 1202 that can have a handle 1203 like the handle 1103 shown in FIGS. 40-42, so as to provide another embodiment of a single tester device 1200, having round-base conical sharp probes 1206, with radial supports 1208 that support the collar 1205 having dull probes 1204. In fact, all of the test heads described and shown herein can be used with a handle to form a single tester device.

Other modifications and implementations will occur to those skilled in the art without departing from the spirit and the scope of the invention as claimed. Accordingly, the above description is not intended to limit the invention, except as indicated in the following claims.

What is claimed is:

1. An allergy skin testing device, the device comprising:
a central post having a gripping portion;
at least one sharp probe extending longitudinally from an end of the central post, the sharp probe being for administering an allergen to skin; and
at least three dull probes substantially surrounding the at least one sharp probe, the at least three dull probes being for activating a pain gate effect in nerves of the skin while the at least one sharp probe administers the allergen to the skin, the dull probes extending further along the central post than the at least one sharp probe extends beyond the central post,
the gripping portion of the central post being for securely grasping the central post so as to facilitate pressing the plurality of dull probes into the skin before pressing the at least one sharp probe into the skin using one continuous movement of the central post towards the skin.

2. The device of claim 1, wherein the dull probes are supported by a support structure that is supported by a longitudinal portion of the central post.

3. The device of claim 1, wherein the at least one sharp probe includes at least one additional sharp probe so as to be a plurality of sharp probes arranged in a regularly spaced array.

4. The device of claim 1, further comprising:
a collar in coaxial relationship with the central post, the dull probes being supported by the collar, the collar being resiliently supported by the central post.

5. The device of claim 4, wherein the collar is supported by a plurality of resilient members that extend from the central post to the collar.

6. The device of claim 4, wherein there is at least one discontinuity in the collar.

7. The device of claim 1, further comprising a collar, wherein the dull probes are formed as bumps on a forward edge of the collar.

8. The device of claim 7, wherein the collar is resiliently supported by the central post.

9. The device of claim 7, wherein the collar is rigidly supported by the central post.

10. The device of claim 1, wherein the dull probes are supported by a flange of the central post, the flange having a surface that is substantially co-planar with the end of the central post.

11. The device of claim 10, wherein the dull probes are evenly spaced around the periphery of the flange.

12. An allergy skin testing device, the device comprising:
a central post having a gripping portion;
a plurality of sharp probes extending longitudinally from an end of the central post, the sharp probes being for administering an allergen to skin; and
a collar substantially surrounding the plurality of sharp probes, the collar having a plurality of dull probes being for activating a pain gate effect in nerves of the skin while the sharp probes administer the allergen to the skin, the dull probes extending further along the central post than the sharp probes extend beyond the central post,
the gripping portion of the central post being for securely grasping the central post so as to facilitate pressing the plurality of dull probes into the skin before pressing the plurality of sharp probes into the skin using one continuous movement of the central post towards the skin.

13. The device of claim 12, wherein the collar is rigidly supported by the central post.

14. The device of claim 12, wherein the collar is resiliently supported by the central post.

15. The device of claim 12, wherein the collar is supported by a flange of the central post, the flange having a surface that is substantially co-planar with the end of the central post.

16. The device of claim 12, wherein the plurality of sharp probes is arranged in a regularly spaced array.

17. An allergy skin testing device, the device comprising:
a central post having a gripping portion;
a plurality of sharp probes extending longitudinally from an end of the central post, the sharp probes being for administering an allergen to skin; and
a flange at the end of the central post, the flange having a plurality of dull probes substantially surrounding the plurality of sharp probes, the dull probes being for activating a pain gate effect in nerves of the skin while the sharp probes administer the allergen to the skin, the dull probes extending further along the central post than the sharp probes extend beyond the central post,
the gripping portion of the central post being for securely grasping the central post so as to facilitate pressing the plurality of dull probes into the skin before pressing the plurality of sharp probes into the skin using one continuous movement of the central post towards the skin.

18. The device of claim 17, wherein the plurality of sharp probes is arranged in a regularly spaced array.

19. The device of claim 17, further comprising:
a collar supported by the flange, the collar having the plurality of dull probes evenly spaced around the circumference of the collar.

20. The device of claim 19, wherein the dull probes are regular undulations running evenly around the circumference of the collar.

21. A multiple allergy skin testing device, the device comprising:
a plurality of central posts;
at least one sharp probe extending longitudinally from an end of each central post, the at leats one sharp probe being for administering an allergen to skin;
a collar substantially surrounding each at least one sharp probe, each collar having a plurality of dull probes being for activating a pain gate effect in nerves of the skin while the at least one sharp probe administers the allergen to the skin, the dull probes extending further along each central post than the at least one sharp probe extends beyond the central post; and
a gripping element connected to each central post, the gripping element being for securely controlling the plurality of central posts so as to facilitate pressing each respective plurality of dull probes into the skin before pressing each respective at least one sharp probe into the skin using one continuous movement of the gripping portion towards the skin.

22. The device of claim 21, wherein each collar is rigidly supported by a respective central post.

23. The device of claim 21, wherein each collar is resiliently supported by a respective central post.

24. The device of claim 21, wherein each collar is supported by a respective flange of a respective central post, the flange having a surface that is substantially co-planar with the end of the respective central post.

25. The device of claim 21, wherein each at least one sharp probe includes at least one additional sharp probe so as to form a plurality of sharp probes arranged in a regularly spaced array.

26. An allergy skin testing device, the device comprising:
a central post;
a plurality of sharp probes extending longitudinally from an end of the central post, the sharp probes being for administering an allergen to skin;
a flat-ended collar having a continuous flat dull edge substantially surrounding the plurality of sharp probes, the flat-ended collar being for activating a pain gate effect in nerves of the skin while the sharp probes administer the allergen to the skin, the flat-ended collar extending further along the central post than the sharp probes extend beyond the central post; and
a gripping portion of the central post, the gripping portion being for securely grasping the central post so as to facilitate pressing the flat-ended collar into the skin before pressing the plurality of sharp probes into the skin using one continuous movement of the central post towards the skin.

27. The device of claim 27, wherein the flat-ended collar is supported by a support structure that is supported by a longitudinal portion of the central post.

28. The device of claim 27, wherein the plurality of sharp probes is arranged in a regularly spaced array.

29. The device of claim 27, wherein the flat-ended collar is resiliently supported by the central post.

30. The device of claim 27, wherein the flat flat-ended collar is rigidly supported by the central post.

31. The device of claim 27, wherein the flat-ended collar is supported by a plurality of members that extend from the central post to the flat-ended collar.

32. The device of claim 27, wherein there is at least one discontinuity in the flat-ended collar.

33. The device of claim 27, wherein the flat-ended collar is coaxially supported by the central post.

34. The device of claim 27, wherein the flat-ended collar is supported by a flange of the central post.

35. A multiple allergy skin testing device, the device comprising:
a plurality of central posts;
a plurality of sharp probes extending longitudinally from an end of each central post, the sharp probes being for administering an allergen to skin;
a flat-ended collar having a continuous flat dull edge substantially surrounding each plurality of sharp probes, each flat-ended collar being for activating a pain gate effect in nerves of the skin while the sharp probes administer the allergen to the skin, the flat-ended collar extending further along each central post than the sharp probes extend beyond the central post; and
a gripping element connected to each central post, the gripping element being for securely controlling the plurality of central post so as to facilitate pressing each respective flat-ended collar into the skin before pressing each respective plurality of sharp probes into the skin using one continuous movement of the gripping portion towards the skin.

36. The device of claim 38, wherein each flat-ended collar is rigidly supported by a respective central post.

37. The device of claim 38, wherein each flat-ended collar is resiliently supported by a respective central post.

38. The device of claim 38, wherein each flat-ended collar is supported by a respective flange of a respective central post.

39. The device of claim 38, wherein each plurality of sharp probes is arranged in a regularly spaced array.
